(19) United States
(12) Patent Application Publication
(10) Pub. No.: US 2008/0161327 A1
(43) Pub. Date: Jul. 3, 2008

(54) Substituted 9-Alkyladenines and the Use Thereof

(75) Inventors: Gevork Minaskanian, Richmond, VA (US); Keith Rippel, Richmond, VA (US)

Correspondence Address: STERNE, KESSLER, GOLDSTEIN & FOX P.L.L.C., 1100 NEW YORK AVENUE, N.W., WASHINGTON, DC, 20005, US

(73) Assignee: Aderis Pharmaceuticals, Inc., Hopkinton, MA (US)

(21) Appl. No.: 11/886,086

(22) PCT Filed: Mar. 10, 2006
(86) PCT No.: PCT/US06/08715

§ 371 Date: Sep. 11, 2007

Related U.S. Application Data
(60) Provisional application No. 60/660,466, filed on Mar. 11, 2005.

(51) Int. Cl. A61K 31/52; C07D 473/32; A61P 9/00

(52) U.S. Cl. 514/263.4; 544/277

(57) ABSTRACT

The invention relates to substituted 9-alkyladenines of Formula I: or a pharmaceutically acceptable salt thereof, wherein R₁-R₆ are defined as set forth in the specification. The invention is also directed to the use of such compounds to inhibit adenosine A₁ receptor activation in a mammal. The compounds of the present invention are useful as diuretics, renal protectives against acute or chronic renal failure, as well as agents to improve the therapeutic outcome resulting from defibrillation or cardiopulmonary resuscitation by preventing post-resuscitation bradycardia, bradyarrythmia and cardioplegia, to restore cardiac function following a cardioplegic procedure, and to treat or prevent intermittent claudication.

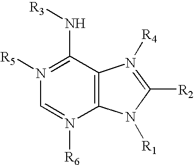

### BACKGROUND OF THE INVENTION

1. Field of the Invention

This invention is in the filed of medicinal chemistry. In particular, this invention relates to novel substituted 9-alkyladenine compounds. These compounds act as adenosine receptor antagonists, specifically adenosine A₁ receptor antagonists. Thus, the present invention also relates to a method of inhibiting adenosine A₁ receptor activation in a mammal in need thereof, comprising administering to said mammal an effective amount of a the substituted 9-alkyladenine compound of the present invention. The compounds of the present invention have a therapeutic use, in particular as diuretics, renal protective agents against acute or chronic renal failure, as well as agents to treat or prevent intermittent claudication, for restoring cardiac function following a cardioplegic procedure, and for improving the therapeutic outcome resulting from defibrillation or cardiopulmonary resuscitation by preventing post-resuscitation bradycardia, bradyarrhythmia and cardioplegia.

2. Related Art

Adenosine (9-β-D-ribofuranosyl-9H-purin-6-amine) was characterized in the late 1920's as having hypotensive and bradycardic activity. Since then, considerable research in the molecular modification of adenosine has led to the general conclusion that cardiovascular activity is limited to analogs having intact purine and β-D-ribofuranosyl rings. Further research has more clearly defined how the activity of these adenosine analogs affected the purinergic receptors in peripheral cell membranes, particularly the adenosine A₁, A₂, and A₃ receptors.

Adenosine antagonists have helped explain the role of adenosine in various physiological processes. Specifically, selective antagonists for the adenosine A₁ receptor were critical in defining the physiological importance of A₁ receptor activation. Non-selective adenosine antagonists, such as caffeine and theophylline, have served as starting points for structure activity research with the goal of discovering A₁ receptor selective antagonists (Shimada, J. et al., J. Med. Chem. 34:466-469 (1991)). An example of such a discovery is 8-(dicyclopropylmethyl)-1,3-dipropylxanthine (Shimada et al., supra). Further xanthine derivatives have been described by Neely in U.S. Patent Application Publication No. 2002/0082269 A1 and Wilson et al. in WO 2004/74247 A2. 8-Substituted purine derivatives have been described by Dudley et al. (U.S. Pat. No. 5,208,240). Other purine derivatives have been described by Peck et al. (U.S. Pat. No. 5,981,524). Further purine derivatives have been described by Lin et al. in U.S. Patent Application Publication No. 2003/0220358. Castelhano et al. (U.S. Patent Application Publication Nos. 2004/0082598 and 2004/0082599) describe 7-deazapurine derivatives as A₁ receptor antagonists.

The search continues for potent and selective adenosine A₁ receptor selective antagonists, useful as pharmacological tools and as therapeutic agents. It is an aim of the present invention to additionally provide adenosine A₁ receptor antagonists that minimally penetrate and distribute in the central nervous system in order to exert peripheral therapeutic actions and, yet, avoid centrally mediated side-effects.

### BRIEF SUMMARY OF THE INVENTION

The present invention is related to novel substituted 9-alkyladenines having the structural Formula I:

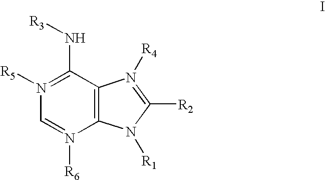

or a pharmaceutically acceptable salt thereof; wherein

R₁ is a straight chain or a branched chain C₁—C₄ alkyl;

R₂ is selected from the group consisting of H, —OR₇, —SR₇, amino, —NH(R₇), —N(R₈)(R₉), aminocarbonyl, halogen, and —CN, where

R₇ is a straight chain or a branched chain C₁—C₆ alkyl and R8 and R₉ are independently a straight chain or a branched chain C₁—C₆ alkyl, or R₈ and R₉ taken together with the N to which they are attached form a 3- to 7-membered heterocycle, said heterocycle optionally including an additional heteroatom which is selected from the group consisting of nitrogen, oxygen and sulfur;

R₃ is selected from the group consisting of:

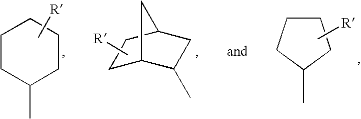

where R′ is H, —OH, or —O-A-R″, where A is a straight or a branched carbon chain of from 1 to 6 carbon atoms and R″ is —SO₃H, —PO₃H, —CO₂H or —N⁺(CH₃)₃;

R₄, R₅, and R6 are each absent (nothing is attached to N) or one of R₄, R₅, or R6 is a straight chain or a branched chain C₁—C₆ alkyl and the other two are absent;

with the proviso that when R₄, R₅, and R6 are each absent, then R′ cannot be H or OH.

The present invention is also related to a pharmaceutical composition, comprising one or more compounds of Formula I in a mixture with one or more pharmaceutically acceptable carriers or diluents. One or more additional pharmaceutically active compounds can also be included in these compositions.

Further, the present invention is related to a method of inhibiting adenosine A₁ receptor activation in a mammal in need thereof, comprising administering to said mammal an effective amount of a compound of Formula I or a pharmaceutically acceptable salt thereof.

In one aspect, the present invention is related to a method of inducing diuresis; protecting the kidneys against acute or chronic renal failure; improving the therapeutic outcome resulting from defibrillation or cardiopulmonary resuscitation; restoring cardiac function following a cardioplegic procedure; or treating or preventing intermittent claudication in a mammal, comprising administering to said mammal in need thereof an effective amount of a compound having the Formula I.

Additional embodiments and advantages of the invention will be set forth in part in the description that follows, and will flow from the description, or may be learned by practice of the invention. The embodiments and advantages of the invention will be realized and attained by means of the elements and combinations particularly pointed out in the appended claims.

It is to be understood that both the foregoing summary and the following detailed description are exemplary and explanatory only and are not restrictive of the invention, as claimed.

### DETAILED DESCRIPTION OF THE INVENTION

Certain novel compounds have now been discovered having activity as adenosine antagonists. These compounds have the structural Formula I:

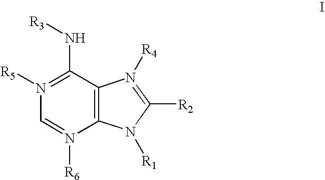

or a pharmaceutically acceptable salt thereof; wherein

R₁ is a straight chain or a branched chain C₁—C₄ alkyl;

R₂ is selected from the group consisting of H, —OR₇, —SR₇, amino, —NH(R₇), —N(R₈)R₉), aminocarbonyl, halogen, and —CN, where

R₇ is a straight chain or a branched chain C₁—C₆ alkyl and R₈ and R₉ are independently a straight chain or a branched chain C₁—C₆ alkyl, or R8 and R₉ taken together with the N to which they are attached form a 3- to 7-membered heterocycle, said heterocycle optionally including an additional heteroatom which is selected from the group consisting of nitrogen, oxygen and sulfur;

R₃ is selected from the group consisting of:

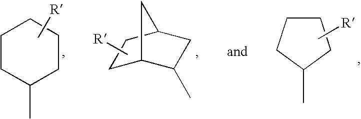

where R′ is H, —OH, or —O-A-R″, where A is a straight or a branched carbon chain of from 1 to 6 carbon atoms and R″ is —SO₃H, —PO₃H, —CO₂H or —N⁺(CH₃)₃;

R₄, R₅, and R6 are each absent (nothing is attached to N) or one of R₄, R₅, or R₆ is a straight chain or a branched chain C₁—C₆ alkyl and the other two are absent;

with the proviso that when R₄, R₅, and R6 are each absent, then R′ cannot be H or OH.

When one of R₄, R₅, or R6 is a straight chain or a branched chain C₁—C₆ alkyl and the others are absent, the compound of Formula I has a positive charge.

Preferably, R₁ is methyl or ethyl, more preferably methyl. Useful compounds include those where R₄, R₅, and R₆ are each absent or one of R₄, R₅, or R6 is a straight chain or branched chain C₁₋₄ alkyl and the other two are absent. Advantageously, one of R₄, R₅, or R6 is methyl or ethyl.

Useful compounds include those where R₂ is hydrogen, halogen, cyano, C₁—C₄ alkoxy, C₁—C₄ alkylthio, amino, —NH(C₁—C₄)alkyl, —N(C₁—C₄ alkyl)(C₁—C₄ alkyl), or heterocycle, such as aziridinyl, piperidinyl, piperazinyl, imidazolidinyl, pyrrolidinyl, imidazolinyl, morpholinyl, pyrazolidinyl, and pyrazolinyl. Advantageously, R₂ is selected from the group consisting of H, halogen, cyano, methoxy, ethoxy, propoxy, thiomethyl, thioethyl, thiopropyl, methylamino, ethylamino, propylamino, isopropylamino, dimethylamino, diethylamino, dipropylamino, di-isopropylamino, ethylmethylamino, methylpropylamino, ethylpropylamino, isopropylmethylamino, ethylisopropylamino, aziridinyl, piperidinyl, piperazinyl, imidazolidinyl, pyrrolidinyl, imidazolinyl, morpholinyl, pyrazolidinyl, and pyrazolinyl. Preferably, R₂ is selected from the group consisting of H, methoxy, ethoxy, propoxy, thiomethyl, thioethyl, thiopropyl, methylamino, ethylamino, propylamino, isopropylamino, dimethylamino, diethylamino, dipropylamino, di-isopropylamino, ethylmethylamino, methylpropylamino, ethylpropylamino, isopropylmethylamino, ethylisopropylamino, piperidinyl, and pyrrolidinyl, and more preferably selected from the group consisting of H, dimethylamino, diethylamino, isopropylmethylamino, isopropylethylamino, piperidinyl and pyrrolidinyl.

Useful compounds include those where R′ is H, OH, —O—(C₁—C₄)alkyl-R″. Advantageously, R′ is H, OH, —(C₁—C₄)alkyl—SO₃H.

In a preferred aspect, R₃ is a substituted cyclohexyl or a substituted norbornyl, wherein the substituent R′ is —O—(C₁—C4)alkyl-SO₃H. Especially preferred compounds are those where R₂ is isopropylmethylamino and R₃ is

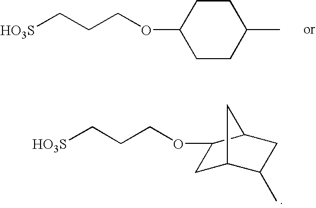

Exemplary useful compounds of the present invention having Formula I include:

(±)-N⁶-[endo-2′-(endo-5′-(3-sulfopropoxy)norbornyl]-8-isopropylmethylamino-9-methyladenine;

(±)-N⁶-[endo-2′-(endo-5′-(4-sulfobutoxy)norbornyl]-8-isopropylmethylamino-9-methyladenine;

(±)-N⁶-[3′-(3-sulfopropoxy)cyclopentyl]-8-isopropylmethylamino-9-methyladenine;

(±)-N⁶-[4′-(4-sulfobutoxy)cyclopentyl]-8-isopropylmethylamino-9-methyladenine;

(±)-N⁶-[endo-2′-(endo-5′-(3-sulfopropoxy)norbornyl]-9-methyladenine;

N⁶-[trans-4′-(³-sulfopropoxy)cyclohexyl]-8-isopropylmethylamino-9-methyladenine;

N⁶-[trans-4′-(4-sulfobutoxy)cyclohexyl]-8-isopropylmethylamino-9-methyladenine;

(±)-N⁶-[endo-2′-(endo-5′-(3-sulfopropoxy)norbornyl]-8-piperidinyl-9methyladenine;

(±)-N⁶-[endo-2′-(endo-5′-(3-sulfopropoxy)norbornyl]-8-pyrrolidinyl-9-methyladenine;

(±)-N⁶-[endo-2′-(endo-5′-(3-sulfopropoxy)norbornyl]-8-dimethylamino-9-methyladenine;

(±)-N⁶-[endo-2′-(endo-6′-(3-sulfopropoxy)norbornyl]-8-isopropylmethylamino-9-methyladenine;

(±)-N⁶-[endo-2′-(endo-5′-(2-sulfoethoxy)norbornyl]-8-isopropylmethylamino-9-methyladenine;

N⁶-[trans-4′-(2-sulfoethoxy)cyclohexyl]-8-isopropylmethylamino-9-methyladenine; and

N⁶-[trans-4′-(3-sulfopropoxy)cyclohexyl]-9-methyladenine;

or a pharmaceutically acceptable salt thereof.

Other exemplary useful compounds include:

N⁶-(endo-5′-hydroxynorbornyl)-7-methyl-8-isopropylmethylamino-9-methyladenine chloride;

N⁶-norbornyl-7-methyl-9-methyladenine chloride;

N⁶-norbornyl-7-methyl-8-isopropylmethylamino-9-methyladenine chloride;

N⁶-(endo-5′-hydroxynorbornyl)-1-methyl-8-isopropylmethylamino-9-methyladenine chloride;

N⁶-norbornyl-1-methyl-9-methyladenine chloride;

N⁶-norbornyl-1-methyl-8-isopropylmethylamino-9-methyladenine chloride;

N⁶-cyclopentyl-1-ethyl-8-isopropylmethylamino-9-methyladenine chloride;

N⁶-(endo-5′-hydroxynorbornyl)-3-methyl-8-isopropylmethylamino-9-methyladenine chloride;

N⁶-norbornyl-3-methyl-9-methyladenine chloride;

N⁶-norbornyl-3-methyl-8-isopropylmethylamino-9-methyladenine chloride;

N⁶-norbornyl-3-hexyl-9-methyladenine chloride;

N⁶-cyclopentyl-3-ethyl-8-isopropylmethylamino-9-methyladenine chloride;

N⁶-cyclopentyl-3-methyl-8-isopropylmethylamino-9-methyladenine chloride; and

N⁶-cyclopentyl-1-methyl-8-isopropylmethylamino-9-methyladenine chloride.

The above compounds are illustrative only and are not meant to be limiting in any way.

Useful halogen groups include fluorine, chlorine, bromine and iodine.

Useful straight chain or branched chain C₁—C₄ alkyl groups include methyl, ethyl, propyl, iso-propyl, butyl, sec-butyl, and tert-butyl groups.

Useful straight chain or branched chain C₁—C₆ alkyl groups include methyl, ethyl, propyl, iso-propyl, butyl, sec-butyl, tert-butyl, pentyl, 3-pentyl, and hexyl groups.

An aminocarbonyl group is —C(O)NH₂.

An amino group is —NH₂.

Useful 3- to 7-membered heterocyclic rings include aziridinyl, piperidinyl, piperazinyl, imidazolidinyl, pyrrolidinyl, imidazolinyl, morpholinyl, pyrazolidinyl, and pyrazolinyl and the like.

The compounds of the present invention are all therapeutically effective adenosine receptor antagonists in mammals. Thus, they are effective for treating conditions that respond to selective adenosine A₁ receptor blockade. Accordingly, the compounds of the present invention are useful as diuretics, renal protective agents against acute or chronic renal failure, as agents to restore cardiac function following a cardioplegic procedure, and to treat or prevent intermittent claudication (angina of the skeletal muscle arising from hypoxia), allergic conditions, autoimmune disorders, ischemia-reperfusion reperfusion organ injury, and as agents to improve the therapeutic outcome resulting from cardiac defibrillation or cardiopulmonary resuscitation by preventing post-resuscitation bradycardia, bradyarrhythmia and cardioplegia.

The invention is also directed to a method of inhibiting adenosine A₁ receptor activation in a mammal in need thereof, comprising administering to said mammal an effective amount of a compound of Formula I or a pharmaceutically acceptable salt thereof.

Some of the compounds disclosed herein may contain one or more asymmetric centers and may thus give rise to enantiomers, diastereomers, and other stereoisomeric forms. The present invention is meant to encompass all such possible forms, as well as their racemic and resolved forms and mixtures thereof. The individual enantiomers may be separated according to methods that are well known to those of ordinary skill in the art. When the compounds described herein contain centers of geometric asymmetry, and unless specified otherwise, it is intended that they include both E and Z geometric isomers. All tautomers are intended to be encompassed by the present invention as well.

As used herein, the term “stereoisomers” is a general term for all isomers of individual molecules that differ only in the orientation of their atoms in space. It includes enantiomers and isomers of compounds with more than one chiral center that are not mirror images of one another (diastereomers).

The term “chiral center” refers to a carbon atom to which four different groups are attached.

The terms “enantiomer” and “enantiomeric” refer to a molecule that cannot be superimposed on its mirror image and hence is optically active wherein the enantiomer rotates the plane of polarized light in one direction and its mirror image rotates the plane of polarized light in the opposite direction.

The term “racemic” refers to a mixture of equal parts of enantiomers and which mixture is optically inactive.

The term “resolution” refers to the separation or concentration or depletion of one of the two enantiomeric forms of a molecule.

The invention disclosed is also meant to encompass all pharmaceutically acceptable salts of the disclosed compounds. Examples of pharmaceutically acceptable salts include inorganic and organic salts. The pharmaceutically acceptable salts include, but are not limited to, halogenides, such as chloride, bromide and iodide, phosphate, sulphate an the like; organic acid salts such as citrate, lactate, tartrate, maleate, fumarate, mandelate, acetate, dichloroacetate, trifluoroacetate, oxalate, formate and the like; and sulfonates such as methanesulfonate, benzenesulfonate, p-toluenesulfonate and the like. Salts are formed, for example, by mixing a solution of a compound of the present invention with a solution of a pharmaceutically acceptable non-toxic acid, such as hydrogen chloride, acetic acid, maleic acid, phosphoric acid and the like.

Compounds of the present invention can be prepared using methods known to those skilled in the art. For example, compounds of the invention wherein R₁ and R₂ are as described above can be prepared by Method A below. X is a leaving group, for example, a halogen. The process is described in detail in Examples 2 and 3.

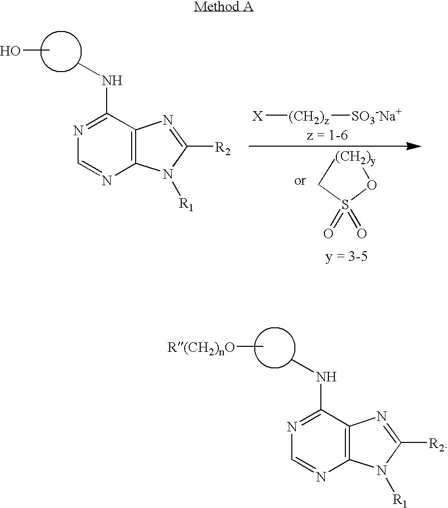

Compounds of the present invention, wherein one of R₄, R₅ or R₆ is a straight chain or a branched chain C₁—C₆ alkyl can be prepared, for example, as shown below in Method B:

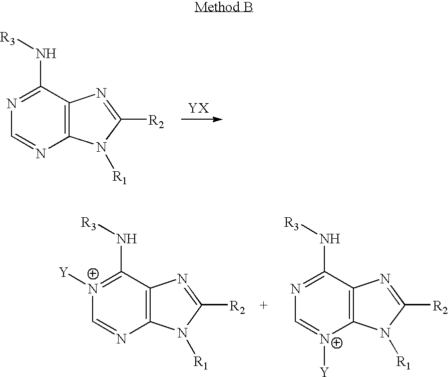

wherein Y is a straight chain or a branched chain C₁—C₆ alkyl and X is a leaving group, for example, halogen. The process is described in detail in Example 4.

The compounds of the present invention were assessed in vitro by human recombinant adenosine receptor binding assays for adenosine antagonist activity. The results are summarized in Example 5. These data were generated to provide measures of the compounds' selectivity (A₁/A_{2A}/A_{2B}/A3) for adenosine A₁ receptors. The compounds of the present invention were also tested for their in vitro functional potency at adenosine A₁ receptors as shown in Example 6.

Compositions within the scope of this invention include all compositions wherein the compounds of the present invention are contained in an amount that is effective to achieve its intended purpose. While individual needs vary, determination of optimal ranges of effective amounts of each component is with the skill of the art. Typically, the compounds may be administered to mammals, e.g., humans, orally at a dose of 0.001 to 100 mg/kg, or an equivalent amount of the pharmaceutically acceptable salt thereof, per day of the body weight of the mammal being treated for diuresis, bradycardia, bradyarrhythmia, and cardioplegia after cardiopulmonary resuscitation or when the compounds are administered as renal protectives. Preferably, about 0.01 to about 10 mg/kg is orally administered to treat or prevent such disorders. For intramuscular injection, the dose is generally about one-half of the oral dose. For example, a suitable intramuscular dose is about 0.0005 to about 50 mg/kg, and most preferably, from about 0.05 to about 5 mg/kg. For intravenous administration, the dose is generally about 0.0001 to about 10 mg/kg, and most preferably, from about 0.001 to about 1 mg/kg.

The unit oral dose can comprise from about 0.1 to about 7000 mg, preferably about 1 to about 700 mg of the compound. The unit dose can be administered one or more times daily as one or more tablets each containing from about 0.1 to about 500, conveniently about 0.1 to 100 mg of the compound or its pharmaceutically acceptable salts or solvates.

In addition to administering the compound as a raw chemical, the compounds of the present invention can be administered as part of a pharmaceutical preparation containing suitable pharmaceutically acceptable carriers comprising excipients and auxiliaries that facilitate processing of the compounds into preparations that can be used pharmaceutically. Preferably, the preparations, particularly those preparations that can be administered orally and that can be used for the preferred type of administration, such as tablets, dragees, and capsules, and preparations that can be administered rectally, such as suppositories, as well as suitable solutions for administration by injection or orally, contain from about 0.01 to 99 percent, preferably from about 0.25 to 75 percent, of active compounds, together with the excipient.

The pharmaceutical compositions of the invention can be administered to any mammal that can experience the beneficial effects of the compounds of the invention. Foremost among such animals are humans, although the invention is not intended to be so limited.

The pharmaceutical compositions of the present invention can be administered by any means that achieve their intended purpose. For example, administration can be by parenteral, subcutaneous, intravenous. intramuscular, intraperitoneal, transdermal or buccal routes. Alternatively, or concurrently, administration can be by the oral route. The dosage administered will be dependent upon the age, health, and weight of the recipient, kind of concurrent treatment, if any, frequency of treatment, and the nature of the effect desired.

The pharmaceutical preparations of the present invention are manufactured in a manner that is, itself, known, for example, by means of conventional mixing, granulating, dragee-making, dissolving, or lyophilizing processes. Thus, pharmaceutical preparations for oral use can be obtained by combining the active compounds with solid excipients, optionally grinding the resulting mixture and processing the mixture of granules, after adding suitable auxiliaries, if desired or necessary, to obtain tablets or dragee cores.

Suitable excipients are, in particular, fillers such as saccharides, for example, lactose or sucrose, mannitol or sorbitol, cellulose preparations and/or calcium phosphates, for example, tricalcium phosphate or calcium hydrogen phosphate, as well as binders, such as starch paste, using, for example, maize starch, wheat starch, rice starch, potato starch, gelatin, tragacanth, methyl cellulose, hydroxypropylmethylcellulose, sodium carboxymethylcellulose and/or polyvinylpyrrolidone. If desired, disintegrating agents can be added, such as the above mentioned starches and also carboxymethyl-starch, cross-linked polyvinylpyrrolidone, agar, or alginic acid or a salt thereof, such as sodium alginate. Auxiliaries are, above all, flow-regulating agents and lubricants, for example silica, talc, stearic acid or salts thereof, such as magnesium stearate or calcium stearate, and/or polyethylene glycol. Dragee cores are provided with suitable coatings that, if desired, are resistant to gastric juices. For this purpose, concentrated saccharide solutions can be used, which may optionally contain gum Arabic, talc, polyvinyl pyrrolidone, polyethylene glycol, and/or titanium dioxide, lacquer solutions and suitable organic solvents or solvent mixtures. In order to produce coatings resistant to gastric juices, solutions of suitable cellulose preparations, such as, acetyl-cellulose phthalate or hydroxypropyl methylcellulose phthalate, are used. Dye stuffs or pigments can be added to the tablets or dragee coatings, for example, for identification or in order to characterize combinations of active compound doses.

Other pharmaceutical preparations which can be used orally include push-fit capsules made of gelatin, as well as soft, sealed capsules made of gelatin and a plasticizer, such as glycerol or sorbitol. The push-fit capsules can contain the active compounds in the form of granules that may be mixed with fillers such as lactose, binders such as starches, and/or lubricants such as talc or magnesium stearate and, optionally, stabilizers. In soft capsules, the active compounds are preferably dissolved or suspended in suitable liquids, such as fatty oils or liquid paraffin. In addition, stabilizers may be added.

Possible pharmaceutical preparations that can be used rectally include, for example, suppositories, which consist of a combination of one or more of the active compounds with a suppository base. Suitable suppository bases are, for example, natural or synthetic triglycerides, or paraffin hydrocarbons. In addition, it is also possible to use gelatin rectal capsules, which consist of a combination of the active compounds with a base. Possible base materials include, for example, liquid triglycerides, polyethylene glycols, or paraffin hydrocarbons.

Suitable formulations for parenteral administration include aqueous solutions of the active compounds in water-soluble form, for example, water-soluble salts. In addition, suspensions of the active compounds as appropriate oily injection suspensions can be administered. Suitable lipophilic solvents or vehicles include fatty oils, for example, ethyl oleate or triglycerides or polyethylene glycol. Aqueous injection suspensions can contain substances that increase the viscosity of the suspension, for example, sodium carboxymethylcellulose, sorbitol, and/or dextran. Optionally, the suspension may also contain stabilizers.

The following examples are illustrative, but not limiting, of the compounds, compositions and methods of the present invention. Suitable modifications and adaptations of the variety of conditions and parameters normally encountered in clinical therapy and which are obvious to those skilled in the art in view of this disclosure are within the spirit and scope of the invention.

### EXAMPLES

### Example 1

The compounds 1-7 listed in Table 1 were prepared using Method A described earlier. The detailed description of the synthesis used for preparing compounds 1-7 is presented in Examples 2 and 3 below.

TABLE 1

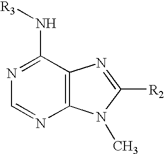

| | | | Elemental | ¹H NMR data |
|---|---|---|---|---|
| Compound | R₂ | R₃ | Formula | (principal peaks) |
| 1 | 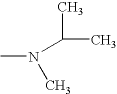 | 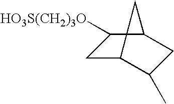 | C₂₀H₃₃ClN₆O₄S(HCl salt) | See below |
| 2 | 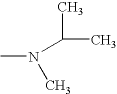 | 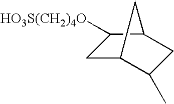 | C₂₁H₃₄N₆O₄S | See below |
| 3 | 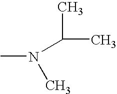 | 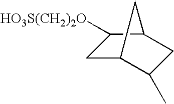 | C₁₉H₂₉N₆O₄S(Na salt) | See below |
| 4 | 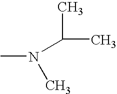 | 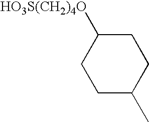 | C₂₆H₄₉N₇O₄S(triethylamine salt) | See below |
| 5 | H | 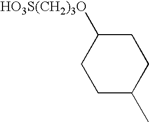 | C₁₅H₂₂N₅O₄S(Na salt) | See below |
| 6 | 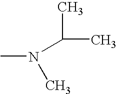 | 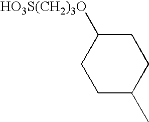 | C₂₅H₄₇N₇O₄S(triethylamine salt) | See below |
| 7 | 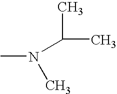 | 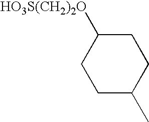 | C₂₄H₄₅N₇O₄S(triethylamine salt) | See below |

¹H NMR data for compounds 1-7 of Table 1 are as follows:

Compound 1: ¹H NMR (300 MHz, CD₃OD): 1.4 (d, 6H), 3.27 (s, 3H), 3.4 (m, 1H), 3.75 (m, 1H), 3.89 (m, 1H), 3.98 (m, 1H), 8.42 (s, 1H);

Compound 2: ¹H NMR (300 MHz, CDCL₃): 1.15 (d, 6H), 2.58 (s, 3H), 2.92 (dt, 2H), 3.3 (bt, 2H), 3.55 (s, 3H), 3.64 (m, 1H), 8.2 (s, 1H);

Compound 3: ¹H NMR (300 MHz, CDCL₃): 1.15 (d, 6H), 2.70 (s, 3H), 3.28 (bt, 2H), 3.48 (s, 3H), 3.55-3.81 (m, 4H), 8.18 (s, 1H);

Compound 4: ¹H NMR (300 MHz, CDCL₃): 1.3 (d, 6H), 2.88 (s, 3H), 2.97 (t, 2H), 3.4 (m, 1H), 3.55 (t, 1H), 2.63 (s, 3H), 3.8 (m, 1H), 4.21 (m, 1H), 8.25 (s, 1H);

Compound 5: ¹H NMR (300 MHz, CD₃OD): 1.5 (m, 2H), 1.82 (m, 4H), 2.05 (m, 2H), 2.15 (m, 2H), 2.9 (t, 2H), 3.6 (m, 1H), 3.78 (s, 3H), 7.92 (s, 1H), 8.2 (s, 1H);

Compound 6: ¹H NMR (300 MHz, CDCL₃): 1.23 (d, 6H), 2.81 (s, 3H), 2.98 (bt, 2H), 3.3 (m, 1H), 3.6 (bs, 5H), 3.98 (m, 1H), 4.19 (bs, 1H), 2.23 (s, 1H); and

Compound 7: ¹H NMR (300 MHz, CDCL₃): 1.23 (d, 6H), 2.81 (s, 3H), 3.62 (s, 3H), 3.78 (m, 1H), 8.3 (s, 1H).

### Example 2

### (35 )-N⁶-[endo-2′-(endo-5′-propoxy-3-sulfonate)-norbornyl]-8-isopropylmethylamino-9-methyladenine (Compound 1)

(±)-N⁶-[endo-2′-(endo-5′-hydroxy)-norbornyl]-8-isopropylmethyl-amino-9-methyladenine (300 mg, 0.909 mmol) was prepared as described in U.S. Pat. No. 5,981,524. This compound was reacted with 100 mg sodium hydride and 1,3-propanesultone (50 mg) in dimethylsulfoxide (3 mL) at 90° C. for 1 hour. After completion of the reaction, solvent was removed by vacuum distillation and the residue was purified using preparative chromatography to isolate the triethylamine salt of the product as follows: Sample Preparation: dissolution in 10 mM triethylamine solvent composed of water/methanol 2:8; Column: Dynamax C18 ( 8 μm, 250 mm×21.4 mm); Mobile Phase A: acetonitrile/water 5:95, Mobile Phase B: water/acetonitrile 10:90 in a step gradient starting with 0% Mobile Phase B and using increasing 5% steps of Mobile Phase B to elute the product, typically at 15% Mobile Phase B; Flow Rate 18 mL/min.

In order to convert the triethylamine salt to the sodium salt of the product, the triethylamine salt was dissolved in water/methanol (9:1) and passed through an ion exchange column (Varian Bondesil-PRS). Appropriate fractions were pooled and concentrated to dryness to result in isolation of 107 mg (24%) of product as the sodium salt.

### Example 3

### N⁶-[(4′-propoxy-3-sulfonate)-cyclohexyl]-9-methyladenine (Compound 5)

6-Chloro-9-methylpurine (12.4 g, 70.5 mmol), trans-4-amino-cyclohexanol (10.0 gm, 87.0 mmol), 250 mL ethanol and triethylamine (33 mL) were mixed in a 500 mL round bottom flask equipped with a condenser and heated to reflux overnight. After cooling, the mixture was concentrated under reduced pressure and the residue washed with CH₂Cl₂/water. The aqueous phase was isolated, concentrated and dried to give an off-white solid. A portion of this product (125 mg, 0.506 mmol) was mixed with sodium hydride (23 mg, 575 mmol), 1,3-propanesultone (200 mg, 1.64 mmol), and 3 mL dimethylsulfoxide, and the mixture was stirred at 70° C. for 1 hour, upon which additional 1,3-propanesultone (200 mg, 1.64 mmol) was added, and the mixture heated again to 70° C. for 30 minutes. The reaction mixture was cooled, concentrated under reduced pressure, and purified using preparative chromatography as described above in Example 2. The resultant triethylamine salt was converted to the sodium salt of the product as described above to give the product as 6.89 mg (25%) of the sodium salt.

### Example 4

### (±)-N⁶-[endo-2′-norbornyl]-8-isopropylmethylamino-1-methyl-9-methyladenine chloride (Compound 17)

### (±)-N⁶-[endo-2′-norbornyl]-8-isopropylmethylamino-3-methyl-9-methyladenine chloride (Compound 13)

(±)-N⁶-[endo-2′-norbornyl]-8-isopropylmethylamino-9-methyladenine (1 g, 3.18 mmol), prepared as described in U.S. Pat. No. 5,981,524, was reacted with methyl iodide (3 mL, 26.1 mmol) in a glass reaction pressure tube with 10 mL acetone. The mixture was heated to 90° C. for 72 hours, upon which a white precipitate was filtered off and washed with acetone. After drying, the crude product was eluted through silica gel (ethyl acetate/methanol 1: 1) to isolate two products, (±)-N⁶-[endo-2′-norbornyl]-8-isopropylmethylamino-1-methyl-9-methyladenine iodide and (±)-N⁶-[endo-2′-norbornyl]-8-isopropylmethylamino-3-methyl-9-methyladenine iodide. After concentration of fractions containing each product, the crude products were purified using preparative chromatography to isolate each product as follows: Sample Preparation: dissolution in water/methanol 1:1; Column: Supelco Amide C16 (250 mm×21.2 mm); Mobile Phase A: acetonitrile/water 5:95, Mobile Phase B: water/acetonitrile 10:90 in a step gradient starting with 0% Mobile Phase B and using increasing 2.5% steps of Mobile Phase B to elute the product, typically at 12.5% Mobile Phase B, Flow Rate: 18 mL/min. The iodide salts were converted to the chloride salts by dissolution in water/methanol (9:1) and elution through an ion exchange column (Varian Bondesil—SAX). Concentration to dryness of appropriate fractions gave the chloride salts of the two products, Compound 17 in a yield of 12.8 mg (11%) and Compound 13 in a yield of 10.4 mg (9%), respectively.

Table 2 presents the compounds 8-17 prepared using the above described methods.

TABLE 2

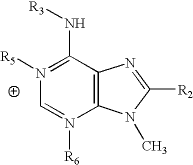

| | | | | | | Elemental |
|---|---|---|---|---|---|---|
| | | | | | | Analysis |
| | | | | | | (calc./obs.) or |
| | | | | | Elemental | ¹H NMR data |
| Compound | R₅ | R₆ | R₂ | R₃ | Formula | (principal peaks) |
| 8 | — | —(CH₂)₅CH₃ | H | 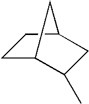 | C₁₉H₃₀N₅(iodide salt) | See below |
| 9 | — | CH₃ | H | 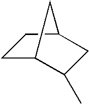 | C₁₄H₂₀N₅(iodide salt) | See below |
| 10 | — | CH₃ | 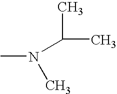 | 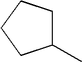 | C₁₆H₂₇N₆(iodide salt) | C: 44.66/44.73H: 6.32/6.40N: 19.53/19.71I: 29.49/29.64 |
| 11 | — | CH₃ | 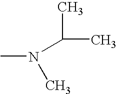 | 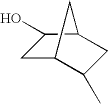 | C₁₈H₂₉N₆O(chloride salt;0.4 moleH₂O) | C: 45.08/45.12H: 6.26/6.03N: 17.52/17.71 |
| 12 | — | —CH₂CH₃ | 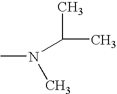 | 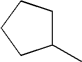 | C₁₇H₂₉N₆(iodide salt) | See below |
| 13 | — | CH₃ | 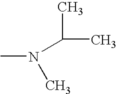 | 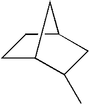 | C₁₈H₂₉N₆(chloride salt) | See below |
| 14 | CH₃ | — | 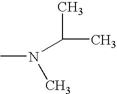 | 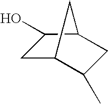 | C₁₅H₂₉N₆O(chloride salt;0.6 moleH₂O) | C: 44.74/44.40H: 6.30/6.02N: 17.39/17.05 |
| 15 | CH₂CH₃ | — | 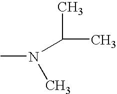 | 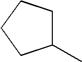 | C₁₇H₂₉N₆(iodide salt) | See below |
| 16 | CH₃ | — | 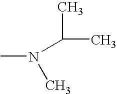 | 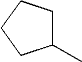 | C₁₆H₂₇N₆(iodide salt) | C: 44.66/44.40H: 6.32/6.23N: 19.53/19.48 |
| 17 | CH₃ | — | 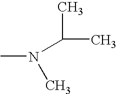 | 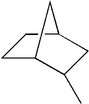 | C₁₈H₂₉N₆(chloride salt) | See below |

¹H NMR data for compounds 8, 9, 12, 13, 15, and 17 of Table 2 are as follows:

Compound 8: ¹H NMR (300 MHz, CDCl₃): 0.88 (t, 3H), 2.9 (s, 3H), 3.65 (s, 3H), 3.9 (m, 1H), 4.85 (m, 1H), 5.11 (m, 1H), 7.52 (s, 1H), 8.1 (s, 1H);

Compound 9: ¹H NMR (300 MHz, CD₃OD): 1.4-1.57 (m, 3H), 1.57-1.74 (m, 4H), 3.85 (s, 3H), 3.95 (s, 3H), 8.28 (s, 1H), 8.51 (s, 1H);

Compound 12: ¹H NMR (300 MHz, CDCl₃): 1.22 (d, 6H), 1.6 (t, 3H), 2.82 (s, 3H), 3.78 (m, 1H), 4.02 (s, 3H), 4.55 (m, 1H), 4.93 (m, 1H), 8.5 (s, 1H);

Compound 13: ¹H NMR (300 MHz, CD₃OD): 1.4 (d, 6H), 3.15 (s, 3H), 3.98 (s, 3H), 4.14 (m, 1H), 4.27 (s, 3H), 4.57 (m, 1H), 8.55 (s, 1H);

Compound 15: ¹H NMR (300 MHz, CDCl₃): 1.25 (d, 6H), 2.9 (s, 3H), 3.68 (s, 3H), 3.92 (m, 1H), 4.98 (dd, 2H), 5.52 (m, 1H), 8.1 (s, 1H); and

Compound 17: ¹H NMR (300 MHz, CD₃OD): 1.3 (d, 6H), 2.95 (s, 3H), 3.72 (s, 3H), 3.95 (s, 3H), 4.05 (m, 1H), 5.35 (m, 1H). 8.4 (s, 1H).

### Example 5

### Receptor Affinity Experiments (Radioligand Binding)

The compounds of the present invention were subjected to in vitro human recombinant adenosine receptor binding assays. The experimental 5 conditions are summarized in Table 3. Scintillation counting was used as the method of detection.

TABLE 3
Experimental Conditions

| Assay | Origin | Ligand | Concentration | | Incubation |
|---|---|---|---|---|---|
| A₁ (h) | CHO | [³H]DPCPX | 1 | nM | 60 min./22° C. |
| | cells | | | | |
| A₁ (h) | CHO | [³H]CCPA | 1 | nM | 60 min./22° C. |
| (agonist | cells | | | | |
| site) | | | | | |
| A_{2A} (h) | HEK-293 | [³H]CGS | 6 | nM | 90 min./22° C. |
| | cells | 21680 | | | |
| A_{2B} (h) | HEK-293 | [³H]MRS | 0.5 | nM | 120 min./22° C. |
| | cells | 1754 | | | |
| A₃ (h) | HEK-293 | [³H]AB- | 0.15 | nM | 90 min./22° C. |
| | cells | MECA | | | |

The IC₅₀ values (concentration causing a half-maximal inhibition of control specific binding) and Hill coefficients (n_{H}) were determined by non-linear regression analysis of the competition curves using Hill equation curve fitting (Motulsky, H. and Christopoulos, A., Fitting Models to Biological Data Using Linear and Nonlinear Regression, GraphPad Software Inc. (2003)).

The inhibition constants (Kᵢ) were calculated from the Cheng Prusoff equation (Motulsky, H. and Christopoulos, A., supra).

Table 4 summarizes the percent inhibition of control specific binding for tested compounds wherein each compound was tested at a concentration of 1×10⁻⁵M.

TABLE 4
Percent Inhibition of Control Specific Binding of the Tested
Compounds

| | Percent Inhibition of Control Specific Binding | | | | |
|---|---|---|---|---|---|
| | | A₁ (h) | | | |
| | | (agonist | | | |
| Compound | A₁ (h) | site) | A_{2A} (h) | A_{2B} (h) | A₃ (h) |
| 11 | 96 | 95 | 31 | 7 | 8 |
| (+isomer) | | | | | |
| 11 | 99 | 97 | 17 | 1 | 1 |
| (−isomer) | | | | | |
| 14 | −1 | 18 | −9 | 5 | 5 |

Tables 5-7 summarize the determined IC₅₀ values, inhibition constants and Hill coefficients for Compound 1. N.C. means that IC₅₀, Kᵢ or n_{H} was not calculable (i.e., the IC₅₀ value is not calculable due to less than 25% inhibition at the highest tested concentration).

TABLE 5

| | A₁ (h) | | | A₁ (h) (agonist site) | | |
|---|---|---|---|---|---|---|
| Compound | IC₅₀ | Kᵢ | n_{H} | IC₅₀ | Kᵢ | n_{H} |
| 1 | 5.2 × 10⁻⁸ | 3.2 × 10⁻⁸ | 0.9 | 2.1 × 10⁻⁷ | 8.5 × 10⁻⁸ | 1.3 |
| (+isomer) | | | | | | |
| 1 | 1.8 × 10⁻⁶ | 1.1 × 10⁻⁶ | 1.0 | 4.3 × 10⁻⁶ | 1.8 × 10⁻⁶ | 1.0 |
| (−isomer) | | | | | | |

TABLE 6

| | A_{2A} (h) | | | A_{2B} (h) | | |
|---|---|---|---|---|---|---|
| Compound | IC₅₀ | Khd i | n_{H} | IC₅₀ | Kᵢ | n_{H} |
| 1 | 3.2 × 10⁻⁶ | 2.6 × 10⁻⁶ | 0.8 | N.C. | N.C. | N.C. |
| (+isomer) | | | | | | |
| 1 | 1.0 × 10⁻⁴ | 8.2 × 10⁻⁵ | 1.4 | N.C. | N.C. | N.C. |
| (−isomer) | | | | | | |

TABLE 7

| | A₃ (h) | | |
|---|---|---|---|
| Compound | IC₅₀ | Kᵢ | n_{H} |
| 1 | >1.0 × 10⁻⁴ | N.C. | N.C. |
| (+isomer) | | | |
| 1 | N.C. | N.C. | N.C. |
| (−isomer) | | | |

Table 8 summarizes the affinity and selectivity values determined for Compound 1.

TABLE 8
The Affinity Ki and A₁ Selectivity vs.
A_{2A}, A_{2B}, and A₃ for Compound 1

| | Affinity Ki (μM) | | | | A₁ Selectivity vs. | | |
|---|---|---|---|---|---|---|---|
| Compound | A₁ | A_{2A} | A_{2B} | A₃ | A_{2A} | A_{2B} | A₃ |
| 1 | 0.032 | 2.6 | >100 | >100 | 81 | >3000 | >3000 |
| (+isomer) | | | | | | | |
| 1 | 1.1 | 82 | >100 | >100 | 75 | >3000 | >3000 |
| (−isomer) | | | | | | | |

These data show the high degrees of adenosine A₁ receptor affinity and selectivity achieved with the tested compounds of the present invention.

### Example 6

### Adenosine A₁ In Vitro Functional Test

A₁ assay—Negative inotropic response (Barrett, R. J. et al., J. Pharmacol. Exp. Ther. 265:227-236 (1993)): Guinea pig left atria were placed in organ baths filled with Krebs Henseleit solution gassed with 95% O₂ and 5% CO₂ and maintained at a temperature of 31° C. An initial resting tension of 1 g was placed on each tissue, which was allowed to equilibrate. Atria were stimulated electrically to produce twitch responses. A fixed concentration of the test compound was added and the tissue was allowed to equilibrate for 1 hour before cumulative additions of an adenosine agonist (either 5′-N-ethylcarboxamidoadenosine (NECA) or N⁶-cyclopentyladenosine (CPA)) to generate a dose response curve. This procedure was repeated with different concentrations of test compounds to generate a family of dose response curves. Schild plots were constructed from the resultant concentration-effect curves, and K_{B} (antagonist dissociation constant) values were obtained. The results presented in Table 9 are expressed as K_{B} values (concentrations of test compound such that one-half of the tissue adenosine receptor population is occupied by the test compound).

TABLE 9
K_{B} (antagonist dissociation constant)
values for Compounds 1, 11, 14, and 6

| Compound | K_{B} (μM) |
|---|---|
| 1 | 32 |
| (+isomer) | |
| 1 | 0.3 |
| (−isomer) | |
| 11 | 1.7 |
| (+isomer) | |
| 14 | 0.5 |
| (−isomer) | |
| 6 | 85 |

The results in Table 9 show the high degrees of functional adenosine A₁ receptor activity achieved with the tested compounds of the present invention.

Having now fully described this invention, it will be understood by those of ordinary skill in the art that the same can be performed within a wide and equivalent range of conditions, formulations and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference herein in their entirety.

1. A compound having the Formula I: 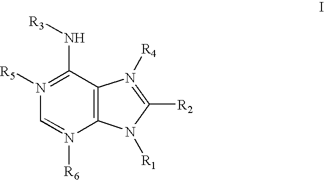
or a pharmaceutically acceptable salt thereof; wherein
R₁ is a straight chain or a branched chain C₁—C₄ alkyl;
R₂ is selected from the group consisting of H, —OR₇, —SR₇, amino, —NH(R₇), —N(R₈)(R₉), aminocarbonyl, halogen, and —CN, where
R₇ is a straight chain or a branched chain C₁—C₆ alkyl and R₈ and R₉ are independently a straight chain or a branched chain C₁—C₆ alkyl, or R₈ and R₉ taken together with the N to which they are attached form a 3- to 7-membered heterocycle, said heterocycle optionally including an additional heteroatom which is selected from the group consisting of nitrogen, oxygen and sulfur;
R₃ is selected from the group consisting of: 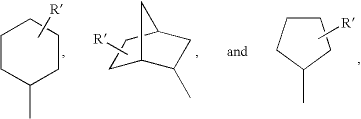
where R′ is H, —OH, or —O-A-R″, where A is a straight or a branched carbon chain of from 1 to 6 carbon atoms and R″ is —SO₃H, —PO₃H, —CO₂H or —N⁺(CH₃)₃;
R₄, R₅, and R6 are each absent (nothing is attached to N) or one of R₄, R₅, or R6 is a straight chain or a branched chain C₁—C₆ alkyl and the other two are absent;
with the proviso that when R₄, R₅, and R6 are each absent, then R′ cannot be H or OH.

2. The compound of claim 1, wherein R₁ is methyl or ethyl.

3. The compound of claim 2, wherein R₁ is methyl.

4. The compound of claim 1, wherein R₂ is selected from the group consisting of hydrogen, halogen, cyano, C₁—C₄ alkoxy, C₁—C₄ alkylthio, amino, —NH(C₁—C₄)alkyl, —N(C₁—C₄ alkyl)(C₁—C₄ alkyl), and a 3- to 7-membered heterocycle selected from the group consisting of aziridinyl, piperidinyl, piperazinyl, imidazolidinyl, pyrrolidinyl, imidazolinyl, morpholinyl, pyrazolidinyl, and pyrazolinyl.

5. The compound of claim 4, wherein R₂ is selected from the group consisting of H, halogen, cyano, methoxy, ethoxy, propoxy, thiomethyl, thioethyl, thiopropyl, methylamino, ethylamino, propylamino, isopropylamino, dimethylamino, diethylamino, dipropylamino, di-isopropylamino, ethylmethylamino, methylpropylamino, ethylpropylamino, isopropylmethylamino, ethylisopropylamino, aziridinyl, piperidinyl, piperazinyl, imidazolidinyl, pyrrolidinyl, imidazolinyl, morpholinyl, pyrazolidinyl, and pyrazolinyl.

6. The compound of claim 5, wherein R₂ is selected from the group consisting of H, methoxy, ethoxy, propoxy, thiomethyl, thioethyl, thiopropyl, methylamino, ethylamino, propylamino, isopropylamino, dimethylamino, diethylamino, dipropylamino, di-isopropylamino, ethylmethylamino, methylpropylamino, ethylpropylamino, isopropylmethylamino, ethylisopropylamino, piperidinyl, and pyrrolidinyl.

7. The compound of claim 6, wherein R₂ is selected from the group consisting of H, dimethylamino, diethylamino, isopropylmethylamino, isopropylethylamino, piperidinyl and pyrrolidinyl.

8. The compound of claim 7, wherein R₂ is isopropylmethylamino.

9. The compound of claim 1, wherein R₃ is: 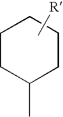
wherein R′ is as defined in claim 1.

10. The compound of claim 1, wherein R₃ is: 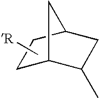
wherein R′ is as defined in claim 1.

11. The compound of claim 1, wherein R′ is H, OH, or —O—(C₁—C₄)R″, wherein R″ is —SO₃H, —PO₃H, —CO₂H or —N⁺(CH₃)₃.

12. The compound of claim 11, wherein R′ is H, OH or —O—(C₁—C₄)SO₃H.

13. The compound of claim 1, wherein R₃ is:

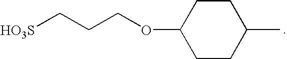

14. The compound of claim 1, wherein R₃ is:

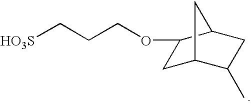

15. The compound of claim 1, wherein R₄, R₅, and R₆ are each absent.

16. The compound of claim 1, wherein one of R₄, R₅, and R6 is methyl or ethyl and the other two are absent.

17. The compound of claim 1, wherein said compound is:
(±)-N⁶-[endo-2′-(endo-5′-(3-sulfopropoxy)norbornyl]-8-isopropylmethylamino-9-methyladenine;
(±)-N⁶-[endo-2′-(endo-5′-(4-sulfobutoxy)norbornyl]-8-isopropylmethylamino-9-methyladenine;
(±)-N⁶-[3′-(3-sulfopropoxy)cyclopentyl]-8-isopropylmethylamino-9-methyladenine;
(±)-N⁶-[4′-(4-sulfobutoxy)cyclopentyl]-8-isopropylmethylamino-9-methyladenine;
(±)-N⁶-[endo-2′-(endo-5′-(3-sulfopropoxy)norbornyl]-9-methyladenine;
N⁶-[trans-4′-(3-sulfopropoxy)cyclohexyl]-8-isopropylmethylamino-9-methyladenine;
N⁶-[trans-4′-(4-sulfobutoxy)cyclohexyl]-8-isopropylmethylamino-9-methyladenine;
(±)-N⁶-[endo-2′-(endo-5′-(3-sulfopropoxy)norbornyl]-8-piperidinyl-9-methyladenine;
(±)-N⁶-[endo-2′-(endo-5′-(3-sulfopropoxy)norbornyl]-8-pyrrolidinyl-9-methyladenine;
(±)-N⁶-[endo-2′-(endo-5′-(3-sulfopropoxy)norbornyl]-8-dimethylamino-9-methyladenine;
(±)-N⁶-[endo-2′-(endo-6′-(3-sulfopropoxy)norbornyl]-8-isopropylmethylamino-9-methyladenine;
(±)-N⁶-[endo-2′-(endo-5′-(2-sulfoethoxy)norbornyl]-8-isopropylmethylamino-9-methyladenine;
N⁶-[trans-4′-(2-sulfoethoxy)cyclohexyl]-8-isopropylmethylamino-9-methyladenine; or
N⁶-[trans-4′-(3-sulfopropoxy)cyclohexyl]-9-methyladenine;
or a pharmaceutically acceptable salt thereof.

18. The compound of claim 1, wherein said compound is a pharmaceutically acceptable salt of
N⁶-(endo-5′-hydroxynorbornyl)-7-methyl-8-isopropylmethylamino-9-methyladenine;
N⁶-norbornyl-7-methyl-9-methyladenine;
N⁶-norbornyl-7-methyl-8-isopropylmethylamino-9-methyladenine;
N⁶-(endo-5′-hydroxynorbornyl)-1-methyl-8-isopropylmethylamino-9-methyladenine;
N⁶-norbornyl-1-methyl-9-methyladenine;
N⁶-norbornyl-1-methyl-8-isopropylmethylamino-9-methyladenine;
N⁶-cyclopentyl-1-ethyl-8-isopropylmethylamino-9-methyladenine;
N⁶-(endo-5′-hydroxynorbornyl)-3-methyl-8-isopropylmethylamino-9-methyladenine;
N⁶-norbornyl-3-methyl-9-methyladenine;
N⁶-norbornyl-3-methyl-8-isopropylmethylamino-9-methyladenine;
N⁶-norbornyl-3-hexyl-9-methyladenine;
N⁶-cyclopentyl-3-ethyl-8-isopropylmethylamino-9-methyladenine;
N⁶-cyclopentyl-3-methyl-8-isopropylmethylamino-9-methyladenine; or
N⁶-cyclopentyl-1-methyl-8-isopropylmethylamino-9-methyladenine.

19. A pharmaceutical composition, comprising the compound as claimed in claim 1 and a pharmaceutically acceptable carrier or diluent.

20. A method for inhibiting adenosine A₁ receptor activation in a mammal in need thereof, comprising administering to said mammal an effective amount of a compound having the Formula I: 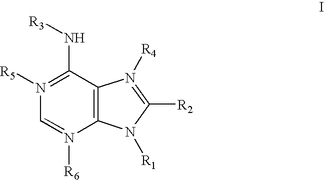
or a pharmaceutically acceptable salt thereof, wherein
R₁ is a straight chain or a branched chain C₁—C₄ alkyl;
R₂ is selected from the group consisting of H, —OR₇, —SR₇, amino, —NH(R7), —N(R₈)R₉), aminocarbonyl, halogen, and —CN, where
R₇ is a straight chain or a branched chain C₁—C₆ alkyl and R₈ and R₉ are independently a straight chain or a branched chain C₁—C₆ alkyl, or R₈ and R₉ taken together with the N to which they are attached form a 3- to 7-membered heterocycle, said heterocycle optionally including an additional heteroatom which is selected from the group consisting of nitrogen, oxygen and sulfur;
R₃ is selected from the group consisting of: 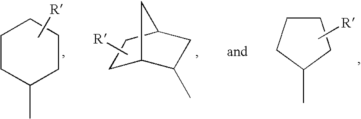
where R′ is H, —OH, or —O-A-R″, where A is a straight or a branched carbon chain of from 1 to 6 carbon atoms and R″ is —SO₃H, —PO₃H, —CO₂H or —N⁺(CH₃)₃;
R₄, R₅, and R6 are each absent (nothing is attached to N) or one of R₄, R₅, or R₆ is a straight chain or a branched chain C₁—C₆ alkyl and the other two are absent;
with the proviso that when R₄, R₅, and R6 are each absent, then R′ cannot be H or OH.

21. (canceled)

22. The method of claim 20, wherein the compound is (±)-N⁶-[endo-2′-(endo-5′-(3-sulfopropoxy)norbornyl]-8-isopropylmethylamino-9-methyladenine or a pharmaceutically acceptable salt thereof.

23. The method of claim 20, wherein the compound is N⁶-[(4′-(3-sulfopropoxy)cyclohexyl]-8-isopropylmethylamino-9-methyladenine or a pharmaceutically acceptable salt thereof.

24. A method of inducing diuresis; protecting the kidneys against acute or chronic renal failure; improving the therapeutic outcome resulting from defibrillation or cardiopulmonary resuscitation; restoring cardiac function following a cardioplegic procedure; or treating or preventing intermittent claudication in a mammal, comprising administering to a mammal in need thereof an effective amount of a compound having the Formula I: 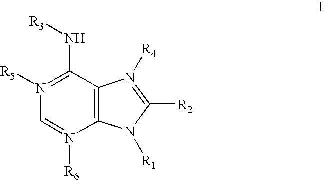
or a pharmaceutically acceptable salt thereof; wherein
R₁ is a straight chain or a branched chain C₁—C₄ alkyl;
R₂ is selected from the group consisting of H, —OR₇, —SR₇, amino, —NH(R₇), —N(R₈)(R₉), aminocarbonyl, halogen, and —CN, where
R₇ is a straight chain or a branched chain C₁—C₆ alkyl and R₈ and R₉ are independently a straight chain or a branched chain C₁—C₆ alkyl, or R₈ and R₉ taken together with the N to which they are attached form a 3- to 7-membered heterocycle, said heterocycle optionally including an additional heteroatom which is selected from the group consisting of nitrogen, oxygen and sulfur;
R₃ is selected from the group consisting of: 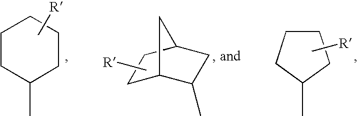
where R′ is H, —OH, or —O-A-R″, where A is a straight or a branched carbon chain of from 1 to 6 carbon atoms and R″ is —SO₃H, —PO₃H, —CO₂H or —N⁺(CH₃)₃;
R₄, R₅, and R6 are each absent (nothing is attached to N) or one of R₄, R₅, or R6 is a straight chain or a branched chain C₁—C₆ alkyl and the other two are absent;
with the proviso that when R₄, R₅, and R6 are each absent, then R′ cannot be H or OH.

25. (canceled)

26. The method of claim 24, wherein the compound is (±)-N⁶-[endo-2′-(endo-5′-(3-sulfopropoxy)norbornyl]-8-isopropylmethylamino-9-methyladenine or a pharmaceutically acceptable salt thereof.

27. The method of claim 24, wherein the compound is N⁶-[(4′-(3-sulfopropoxy)cyclohexyl]-8-isopropylmethylamino-9-methyladenine or a pharmaceutically acceptable salt thereof.
